(19) United States
(12) Patent Application Publication
(10) Pub. No.: US 2022/0151619 A1
(43) Pub. Date: May 19, 2022

(54) ANASTOMOSING STENT AND METHODS OF USE

(71) Applicant: The Board of Supervisors of Louisiana State University and Agricultural and Mechanical College, Baton Rouge, LA (US)

(72) Inventor: Ian Alexander HODGDON, New Orleans, LA (US)

(21) Appl. No.: 17/438,373

(22) PCT Filed: Mar. 11, 2020
(86) PCT No.: PCT/US2020/022016

§ 371 Date: Sep. 10, 2021

Related U.S. Application Data
(60) Provisional application No. 62/816,317, filed on Mar. 11, 2019.

(51) Int. Cl. A61B 17/11; A61L 31/00; A61L 31/14; A61L 31/04

(52) U.S. Cl. CPC A61B 17/1114; A61L 31/005; A61B 2017/00004; A61L 31/044; A61L 31/048; A61L 31/148

(57) ABSTRACT

This invention is directed to an anastomosing stent comprising an internal frame and an external casing, and methods of use thereof.

This application claims priority from U.S. Provisional Application No. 62/816,317, filed on Mar. 11, 2019, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to an anastomosing stent comprising an internal frame and an external casing, and methods of use thereof.

### BACKGROUND OF THE INVENTION

Bowel resection is a common surgical procedure. More than one million bowel resections are performed annually in the United States alone: 600,000 colon resections preformed per year in 2015 (SAGES Data), 400,000 new esophageal cancers diagnosed per year, 179,000 gastric bypass procedures performed in 2013, and small bowel resection is common in trauma, inflammatory bowel disease, adhesions, fistula, and bowel obstruction conditions. Bowel resection is also used to treat many congenital conditions within the pediatric population. The leak rate from bowel anastomosis is between 3-26%, with $28.6 million in additional cost per 1,000 post-op colon resection patients in the first 30 days with leaks. It is a conservative estimate that hundreds of millions of dollars are spent annually to address the complications of bowel leaks.

Enteric anastomosis are common procedures for thoracic, general, laparoscopic, and colorectal surgeons. Leaks from esophageal, small bowel and colon are a major source of morbidity and mortality with a mortality rate of 6-39%. In addition to death, leaks lead to sepsis, abscesses, fistulas, and the anastomosis being taken down and an ostomy being performed. Some patients are at such a high risk for enteric leak that creating an ostomy is a safer procedure. One common example is perforated diverticulitis, and these patients live with ostomies for months to years. Collectively, these interventions add a major financial cost to patients, hospitals, and the health care system.

### SUMMARY OF THE INVENTION

The present invention provides an anastomosing stent. In embodiments, the anastomosing stent comprises an internal frame and an external casing. In embodiments, the external casing substantially covers the internal frame. The stent can further comprise a cavity or lumen extending therethrough along a longitudinal axis.

In embodiments, the stent substantially or completely dissolves over a period of time when implanted in a subject. For example, the period of time comprises about 1 week, about 2 weeks, about 3 weeks, about 1 month, about 2 months, about 3 months, about 6 months, or longer than 6 months.

In embodiments, the internal frame dissolves in about 2 hours, about 4 hours, about 8 hours, about 16 hours, about 24 hours, or longer than 24 hours.

In embodiments, the external casing dissolves in about 1 week, about 2 weeks, about 3 weeks, about 1 month, about 2 months, about 3 months or longer than 3 months.

In embodiments, the stent comprises one or more layers of the external casing. For example, the stent comprises 4 or more layers of the external casing. For example, the stent comprises between 4 layers and 15 layers of external casing.

In embodiments, the stent comprises no more than 20 layers of the external casing. For example, the stent comprises no more than 15 layers of the external casing.

In embodiments, the stent comprises 8 layers of the external casing.

In embodiments, the thickness of the internal frame comprises about 1 mm to about 2 mm.

In embodiments, the thickness of the external casing comprises about 1 mm to about 4 mm.

In embodiments, the internal frame is constructed of a biocompatible material. For example, the biocompatible material is a non-toxic material or is a biodegradable material. Non-limiting examples of such materials comprise polyvinyl alcohol (PVA), starched polyglactin (vicryl), collagen, magnesium, plant based fiber, or any combination thereof

In embodiments, the material substantially or completely dissolves over a period of time when implanted in a subject. For example, the period of time comprises no more than about 2 hours, no more than about 3 hours, no more than about 6 hours, no more than about 12 hours, or no more than about 24 hours.

In embodiments, the material comprises an inert material, such as a fiber. The fiber can be a plant-based fiber.

In embodiments, the internal frame is 3D printed.

In embodiments, the external casing is constructed of a biocompatible material. For example, the biocompatible material is a non-toxic material. The biocompatible material can comprise a biodegradable material which substantially or completely dissolves over a period of time when implanted in a subject. For example, the period of time comprises about 1 week, about 2 weeks, about 3 weeks, about 1 month, about 2 months, about 3 months, or longer than 3 months.

In embodiments, the external casing comprises a biological membrane. The biological membrane can comprise an acellular or substantially acellular biological membrane. The biological membrane can comprise a decellularlized or substantially decellularized biological membrane.

In embodiments, the biological membrane comprises mammalian connective tissue and/or basement membrane. For example, the connective tissue comprises mammalian submucosa or amniotic membrane. For example, the mammalian submucosa comprises porcine submucosa, human submucosa, or bovine submucosa. In one embodiment, the biological membrane comprises dermis, pericardium, blood vessel, or plant-based material.

In embodiments, the stent comprises a synthetic material, an absorbable material, a non-porous material, or a combination thereof.

In embodiments, the stent comprises an enteric stent.

In embodiments, the stent is configured to be implanted using an endoscopic balloon.

In embodiments, the stent is configured to be affixed to a site of anastomosis. For example, the stent comprises a diameter about 22 mm to about 60 mm. The diameter can be about 32 mm. In exemplary embodiments, the stent comprises a length of about 1 cm to about 30 cm. In exemplary embodiments, the stent comprises a length of about 5 cm to about 10 cm.

In embodiments, the stent is flexible, semi-flexible, or rigid.

The stent can include a curved region or an angled region.

In embodiments, the stent is “C” shaped. In other embodiments, the stent is “Y” shaped.

In embodiments, the stent is configured to be a bioscaffold. In exemplary embodiments, the bioscaffold is populated with viable cells. The bioscaffold can be populated with any cell that is viable. The term “viable cell” can refer to a cell that is alive and capable of growth, proliferation, migration, and/or differentiation. A viable cell can be a living cell. Exemplary viable cells comprise epithelial cells.

Aspects of the invention are further directed towards a method of implanting within a subject the anastomosing stent as described herein. In embodiments the method comprises obtaining the stent. The method can comprise implanting the stent to a site in the subject, thereby implanting in the subject the stent. For example, the site can comprise a site of anastomosis, such as surgical anastomosis.

Still further, aspects are directed towards a method for preventing anastomotic leakage. In embodiments, the method comprises implanting within a subject known to have or at risk of having an anastomosis the stent as described herein, thereby preventing leakage.

In embodiments, the stent is implanted under the site of anastomosis. By way of example, the stent can be implanted to reinforce, repair or cover a defect. For example, the stent can be implanted to reinforce, repair or cover a leak, a fistula, a stricture, or a combination thereof.

In embodiments, the stent is implanted under, over, or between a defect, such as under or over the site of anastomosis.

In embodiments, the stent is affixed to the site of anastomosis by glue, staples, pressure, sutures, or clips.

In embodiments, the anastomotic leakage is in the intestine (i.e., colon), esophagus, stomach, rectum, bile duct, ureter, or urethra. For example, the cause of the anastomotic leakage is surgery, such as tissue resection.

In embodiments, the stent can be placed in any tubular structure, including but not limited to veins, arteries, small bowel, trachea, and bronchus.

In embodiments, the stent is implanted using an endoscopic balloon.

Further, aspects of the invention are directed towards a method of making a stent. In embodiments, the method comprises obtaining an internal frame and an external casing, wherein the external casing comprises a biological membrane; placing the external casing over the internal frame, wherein the external casing substantially covers the internal frame. The stent can further comprise a cavity or lumen extending there through along a longitudinal axis; dehydrating the biological membrane to produce a stent capable of being stored for a period of time.

In embodiments, the dehydrated stent is rehydrated in water or saline solution prior to implanting in the subject.

Still further, aspects of the invention are directed towards a kit comprising an anastomosing stent described herein. In embodiments, the kit comprises an internal frame and an external casing. In embodiments, the external casing comprises a biological membrane.

In embodiments, the kit can further comprise a solution for rehydrating the biological membrane and/or stent.

Other objects and advantages of this invention will become readily apparent from the ensuing description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A shows a top view of a collagen stent under one embodiment (bottom) and two separate sections of cadaveric porcine intestines that are aligned for anastomosis at the (top).

FIG. 1B shows a top view of a collagen stent under another embodiment (bottom) and two separate sections of cadaveric porcine intestines that are aligned for anastomosis at the (top).

FIG. 1C shows a close-up cross sectional view of a collagen stent under one embodiment.

FIG. 1D provides a top view of one end of a collagen stent under one embodiment.

FIG. 1E shows an alternative top view of a collagen stent under an embodiment.

FIG. 1F shows a cross-sectional view of a collagen stent with a cadaveric porcine intestine attached thereto.

FIG. 2A shows a PVA internal frame component of stent under one embodiment.

FIG. 2B provides an internal frame component of a stent under another embodiment with perforations and a multilayered covering.

FIG. 3A shows a cross-sectional, perspective view of an internal frame of a stent with a lattice structure under one embodiment.

FIG. 3B provides a cross-sectional, perspective view of a stent under another embodiment of the present invention. An external casing can be seen surrounding the internal frame of the stent.

FIG. 3C shows a top view of a C-shaped embodiment of the presently disclosed anastomosing stent.

FIG. 3D shows a top view C-shaped embodiment comprised of an alternative material.

FIG. 3E provides side perspective view of a stent under yet another embodiment. A thick internal frame can be seen underneath an external casing.

FIG. 3F shows a top view of two alternative stents lying parallel to one another. One stent includes a flanged end (left) while the other lacks a flanged end (right). In embodiments, the stents can be collagen stents. In studies described herein, the stents are polyvinyl alcohol (PVA) internal frame with porcine submucosa covering.

FIG. 4A shows an anastomosing stent under one embodiment being placed within a first bowel tissue in preparation for anastomosis.

FIG. 4B shows the anastomosing stent of FIG. 4A with a second bowel tissue arranged over the stent and aligned for anastomosis with the first bowel tissue.

FIG. 4C shows the first and second bowel tissues of FIG. 4B following anastomosis using the stent of FIG. 4A.

FIG. 5A shows an opened bowel following anastomosis to reveal the internal casing under one embodiment of the present invention. The casing is shown attached to the interior wall the bowel. Sutures were placed through the bowel wall and placed into either the outer or both outer and inner layer.

FIG. 5B provides a partially opened bowel following anastomosis to reveal the external casing under one embodiment. The external casing is shown attached to the interior wall of the bowel and residing within the lumen of the bowel.

FIG. 6A shows schematics of one embodiments of the present invention.

FIG. 6B shows schematics of another embodiment of the present invention.

FIG. 6C provides schematics of a C-shaped stent under an alternate embodiment of the present invention.

FIG. 8 shows the stent of the 2-week study of Example 3. Stent used which placed 8 layers of SIS over a PVA stent.

FIG. 9 shows a 2 cm defect in the anastomosis over the stents. Previous studies in pigs showed that leaving a 2 cm opening in the colon wall would lead to 100% leak rate. A 2 cm defect was left but a stent was sewed under. The results were that there was no leakage in 5/5 pigs at over 2 weeks, and no other complications from stent such as infection, bleeding, obstruction.

FIG. 10 shows an anastomosing stent under one embodiment.

FIG. 11 shows healing of the colon during a study using an anastomosing stent under one embodiment.

FIG. 12 shows sewing in the anastomosing stent and leaving a 2 cm hole. The area between the 2 blue sutures is the area that healed without leaking.

### DETAILED DESCRIPTION OF THE INVENTION

Surgical anastomosis, such as of the esophagus, intestine, stomach, colon and rectum, have a high incidence of leak after resection. Leaks lead to death, morbidity, increased cost and ostomy. Intestinal, rectal, and anal fistula between the skin and other organs have been treated with permanent stents, but these stents have to be removed, can migrate, and are not flexible enough to be placed around tight corners. Further, current stents generally do not comprise an external casing, and thus do not prevent anastomosis leakage. Placing a slowly absorbable stent with an external casing would prevent the unwanted side effects by preventing leakage while the anastomosis heals without the issues of a permanent stent. Thus, aspects of the invention are directed towards compositions and methods for preventing leakage while the anastomosis heals. Among the embodiments described herein are an anastomosing stent comprising an internal frame and an external casing, wherein the external casing substantially covers the internal frame. The stent can further comprise a cavity or lumen extending there through along a longitudinal axis. The stent substantially or completely dissolves over a period of time, and thus is not affected by the issues of a permanent stent.

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

Abbreviations and Definitions

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

For purposes of the present disclosure, it is noted that spatially relative terms, such as “up,” “down,” “right,” “left,” “beneath,” “below,” “lower,” “above,” “upper” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device can be otherwise oriented (rotated 90 degrees or at other orientations) and the spatially relative descriptors used herein interpreted accordingly.

The terms “subject” and “patient” as used herein can include all members of the animal kingdom including, but not limited to, mammals, animals (e.g., cats, dogs, horses, swine, etc.) and humans.

The term “tissue” as used herein can refer to any conglomeration of cells along with the extracellular matrix that work in concert to carry out a specific function. In embodiments, tissue includes nervous tissue, epithelial tissue, connective tissue, muscular tissue, or a combination thereof. Tissue can include dermis, epidermis, subcutaneous fat, fascia, or any combination thereof. In certain embodiments, the tissue may be injured or diseased. Injured or diseased tissue can refer to any tissue that is inflamed, dry, cancerous, wounded, abraded, eroded, burned, fractionated, or has been subjected to any other type of tissue injury or disease or combinations thereof. Injured or diseased tissue can refer to any of various skin conditions known in the art.

As used herein, the term “biodegradable” and its variants can refer to degradation or general breakdown of material in vivo. As used herein, the term “bioabsorbable” and its variants can refer to degradation or general breakdown and metabolism of material in vivo.

Anastomosing Stent

Aspects of the invention are directed towards an anastomosing stent. In various embodiments, the stent comprises an internal frame, an external casing, or a combination thereof. In embodiments, the external casing substantially covers the internal frame, and a cavity or lumen extends through the stent along a longitudinal axis. Materials useful for the stent (e.g., the external casing and/or internal frame) can be any of a variety of biologic or synthetic materials.

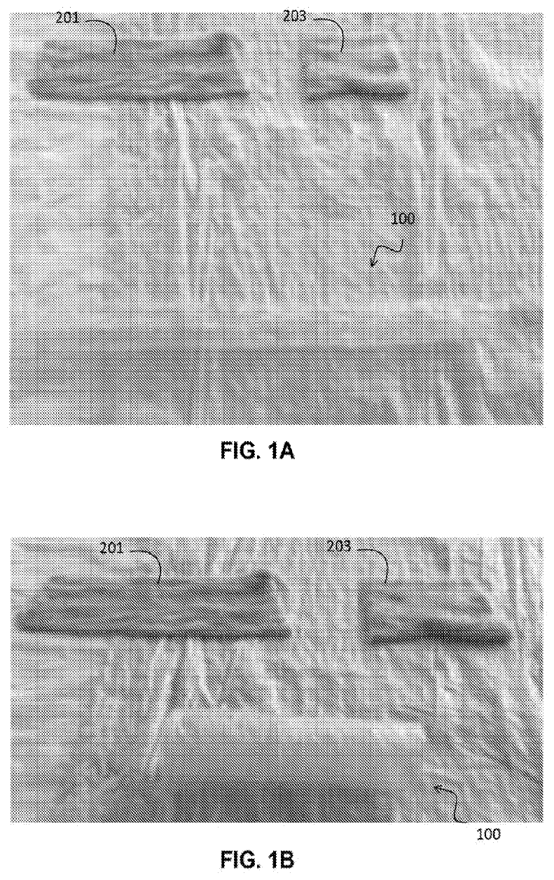

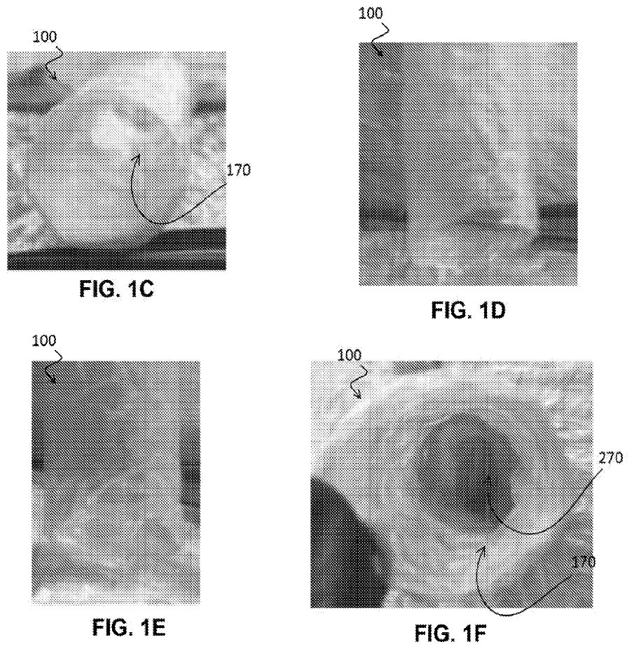

Referring to FIGS. 1A-1F, the stent 100 can be substantially cylindrical, comprising two ends with a lumen or cavity 170 extending longitudinally through the stent 100.

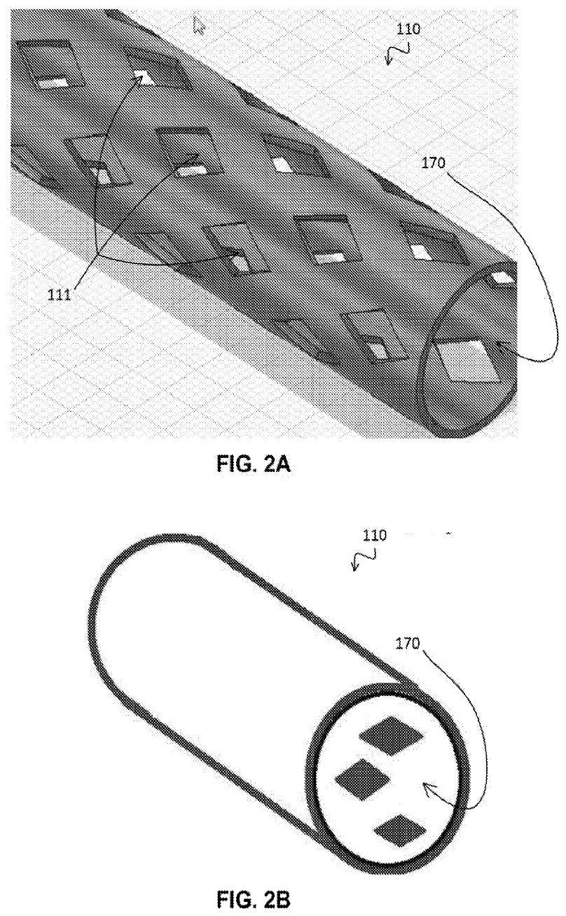

As shown in FIGS. 2A and 2B, the stent can comprise an internal frame 110. In embodiments, the internal frame 110 comprises a lattice structure (FIG. 2A) with opening or pores 111 extending through the walls of the frame. Alternatively, as shown in FIG. 2B, the internal frame 110 can comprise a solid frame.

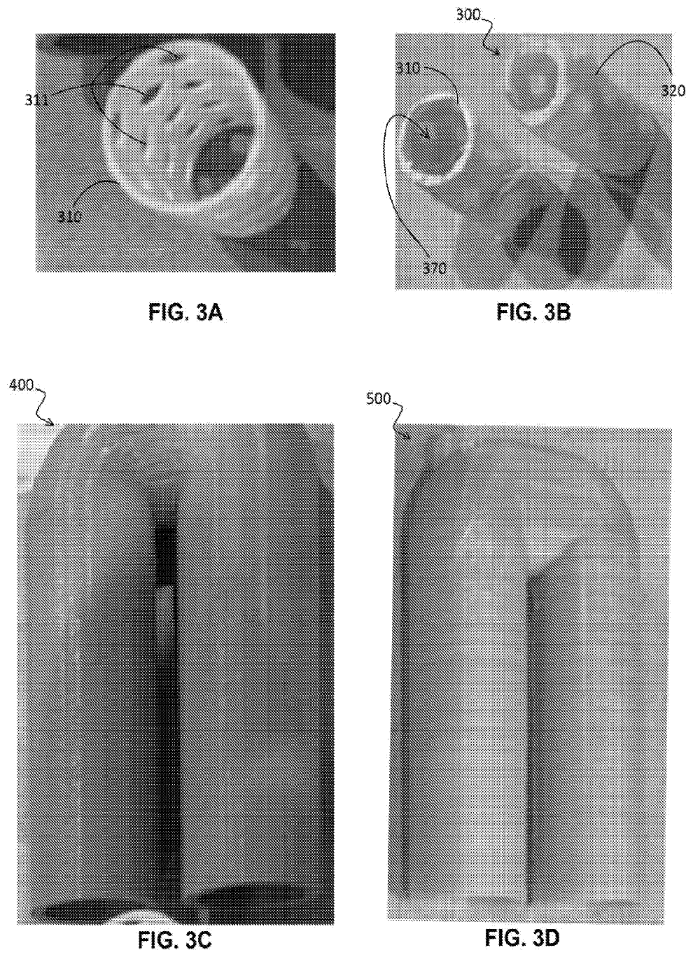

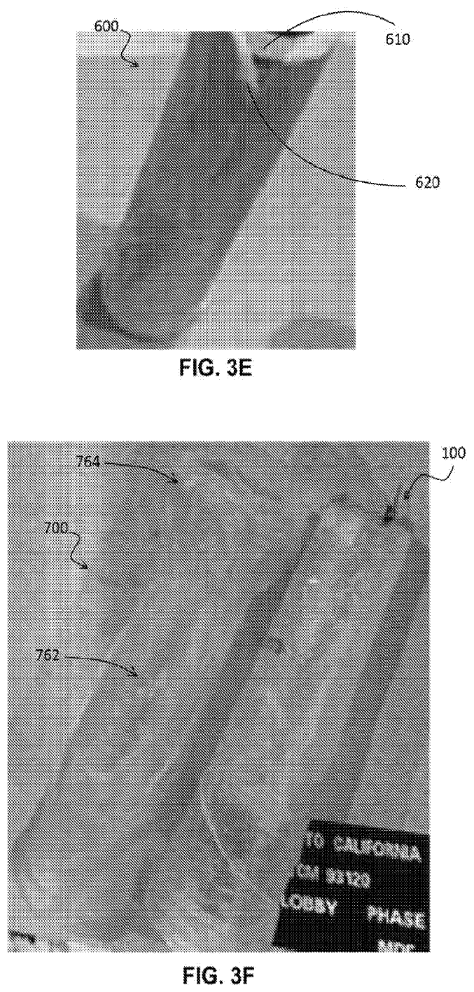

As shown in FIGS. 3A-3F, the stent 100, 300, 400, 500, 600, 700 can comprise various exemplary shapes, configurations, and sizes and can be comprised of any suitable material known in the art. As clearly seen in FIGS. 3B and 3E, the stent 300, 600, can comprise an external casing 320, 620 that at least partially covers the internal frame 310, 610. In embodiments, the external casing 320, 620 covers the entire external surface of the internal frame. The external casing 320, 620 can cover only a portion of the internal frame. In certain embodiments, the external casing 320, 620 covers the ends of the internal frame 310, 610, the middle of the internal frame 310, 610, or a combination thereof. The external casing 320, 620 can cover at least a portion of the external surface of the frame 310, 610, at least a portion of the internal surface of the frame 310, 610 (and thus, line the lumen or cavity of the stent), or a combination thereof. In embodiments, the external casing 320, 620 substantially covers the external surface of the frame 310, 610, the internal surface of the frame 310, 610, or combination thereof. In embodiments, the external casing can be on any or all sides of the internal frame, including but not limited to covering the internal surface of the stent.

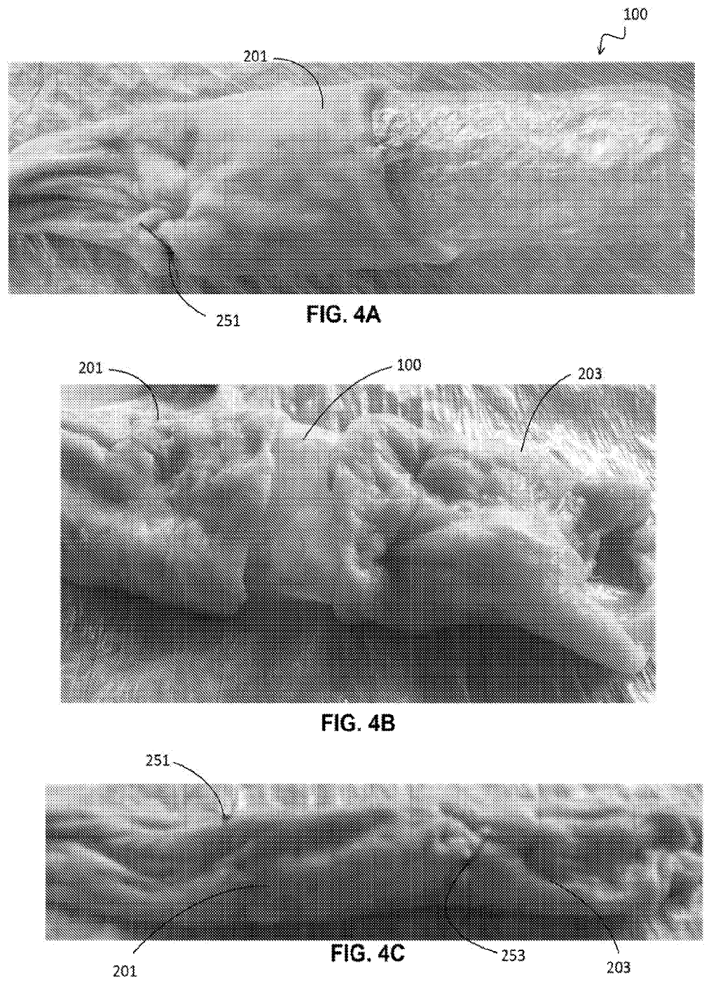

FIGS. 4A-4C provide a series of images that move sequentially through implantation of an anastomosing stent 100 under one embodiment. In FIG. 4A, the stent 100 is shown being inserted in to one end of a first bowel tissue 201. A suture 251 can be seen, which serves as an exemplary means for anchoring the stent 100 to the first bowel tissue 201.

Although suture 251 is used in the present embodiment, any means known in the art is can be used for adhering the stent to tissue. In FIG. 4B, a second section of bowel tissue 203 is shown over the stent 100. One end of the first bowel tissue 201 is aligned next to an end of the second bowel tissue 203 in preparation for anastomosis. FIG. 4C shows the first bowel tissue 201 and the second bowl tissue 203 secured to one another and the stent following anastomosis under one embodiment. As can be seen, both the first 201 and the second 203 bowel tissue are attached to the stent via fasteners 251, 253.

If a permanent stent is implanted in a subject and becomes dislodged, the permanent stent would require surgical removal. Otherwise, the permanent stent may become infected and cause bleeding. However, the stent of the present invention can be formed from a biodegradable material, and optionally bioabsorbable material (such as magnesium or materials mentioned herein), so that the stent substantially or completely dissolves over a period of time when implanted in a subject.

The skilled artisan will recognize that the period of time of degradation and/or absorption of components of the stent can vary. For example, the entirety of the stent can degrade and/or be absorbed over a period of time of about 1 week, 2 weeks, 3 weeks, 1 month, 2 months, or 3 months. If the stent remains in place for too long, such as is the case with permanent stent, the subject can have unwanted medical issues, such as bleeding or infection.

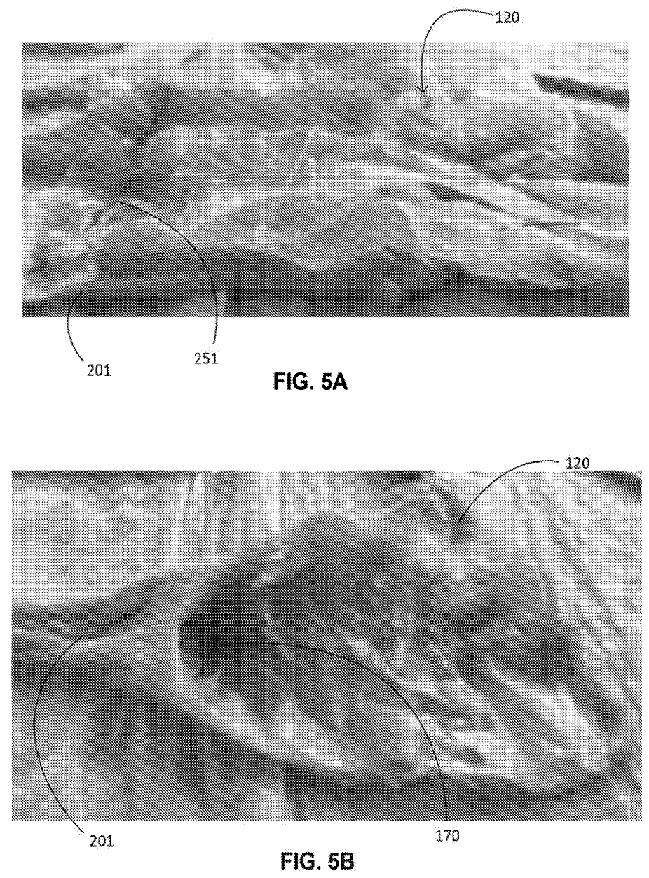

More particularly, the components of the stent can degrade at different periods of time. For example, the internal frame 110 can degrade shortly after the stent is implanted, leaving the external casing 120 to prevent anastomotic leakage and/or promote healing of the anastomosis. FIG. 5 shows a section of bowel 201 with an external casing 120 attached to the interior surface of the bowl 201. As can be seen, in the external casing 120 of the FIG. 5 embodiment forms a lining that prevents leakage at the site of anastomosis when in operation.

The external casing can degrade thereafter. Leaving certain firm materials implanted in a subject, such as the internal frame, can lead to obstruction, erosion, bleeding, and/or perforation. Typically, the internal frame degrades and/or is absorbed faster than the external casing. For example, the internal frame can degrade over a period of time of about less than 12 hours, about 12 hours, or about 24 hours, and the external casing can degrade over a period of time of about 2 weeks, about 1 month, or longer than 1 month. In embodiments, the stent is not permanent.

Any one or more components of the stent can be configured to undergo surface degradation, bulk degradation, or a combination of both. When undergoing surface degradation, the exterior surface of the stent component is progressively broken down until the stent component is completely degraded, resulting in a reduction of the physical size of the stent component as the outer layer dissolves. In bulk degradation, both the exterior surface and the interior of the stent component material erode simultaneously. Thus, when undergoing bulk degradation, the volume of the stent component remains fairly consistent until the material is almost fully degraded.

One or more components of the stent (i.e., the internal frame and/or external casing) of the present invention can be constructed from one or more biocompatible materials. The term “biocompatible” can refer to a material which is not toxic, not injurious or not inhibitory to mammalian cells, tissues, or organs with which it comes in contact. For example, a biocompatible material does not induce an immunological or inflammatory response sufficient to be deleterious to the subject's health or to engraftment of the graft. With biological grafts, there can be in growth and remodeling of tissue. As discussed herein, the biocompatible material can be biodegradable or bioabsorbable, which substantially or completely dissolves over a period of time when implanted in a subject.

Any one or more components of the stent can be constructed from one or more biological materials. For example, the external casing can comprise a biological membrane, which can refer to a sheet or layer of a biological tissue or biological material. Non-limiting examples of a biological materials comprise a mammalian connective tissue and/or basement membrane, such as mammalian submucosa or amniotic membrane. Generally, the biological material can comprise a mammalian tissue, such as submucosa (e.g., porcine submucosa, human submucosa, bovine submucosa), dermis, pericardium collagen or blood vessel. In embodiments, the biological material can also comprise plant-based materials, including those that dissolve. Non-limiting examples include plant-based fibers, such as cardboard or paper, or wood pulp products, such as starched paper or cardboard. The components of the stent can also be constructed from natural materials, such as magnesium.

As described herein, a component of the stent can be configured to be populated by viable cells. However, components of the stent, such as the biological membrane, can be provided as an acellular or substantially acellular material, or a decellularlized or substantially decellularized biological membrane. Acellular porcine submucosa has been used to repair bowels and hernia in animals and humans. An acellular stent or a substantially acellular stent may be provided, for example, to limit immune reaction by the subject to the stent or prevent a stent-associated infection. For example, the body attacks foreign cells and can break them down too quickly, which can cause an immune reaction. Further, living cells can be associated with allergens, which can cause an allergic reaction. Providing an acellular stent can also limit the risk of infection.

The external casing can act as a permeable, selectively permeable, or impermeable membrane. For example, the external casing of the stent will keep the intestinal contents (stool) from leaking through the bowel as a bowel anastomosis heals.

The components of the stent 100 can comprise a synthetic material. For example, the internal frame 110 and/or the external casing 120 can be made of a synthetic mesh. The internal frame 110 and/or the external casing 120 can be made entirely of a one-piece continuous mesh. Referring to FIG. 2, for example, the internal frame 110 can be a one-piece lattice (FIG. 2A) or a one piece solid frame (FIG. 2B). In other embodiments, the internal frame can be of synthetic material and the external casing can be of a different type of a synthetic material or of a biologic material. This may facilitate the components degrading at different time periods. Components of a multi-piece or multi-material stent can be pre-attached or pre-assembled, e.g., attached during manufacture, so a surgeon is not required to spend significant time cutting, connecting, or otherwise assembling the pieces of a stent prior to a surgical installation procedure.

A synthetic stent can be in any form, such as a continuous, solid, or semi-continuous (e.g., perforated) film; or in the form of combined fibers or strands, e.g., a braided, knit, tied, mesh, woven, non-woven, or fabric-type of material; or combinations of these. Certain embodiments of stents include a synthetic portion in the form of a polymeric mesh material. The mesh material includes one or more woven, knit, or inter-linked polymeric filaments or fibers that form multiple fiber intersections or “junctions” throughout the mesh. The fiber junctions may be formed via weaving, knitting, braiding, knotting, joining, ultrasonic welding, use of an adhesive, or other junction-forming techniques, including combinations thereof, leaving openings or pores (“interstices”) between elements of the connected fibers. The size of the pores may be sufficient to allow tissue in-growth and fixation within surrounding tissue upon implantation.

A synthetic stent material can be any synthetic material that can be useful in an implantable surgical device such as a biocompatible polymeric material or a biocompatible non-polymeric synthetic material. Examples of a useful polymeric material comprises polyvinyl alcohol (PVA). Examples of useful polymeric materials that may be useful in a porous material include thermoplastic polymeric materials such as polyolefins (e.g., polypropylenes), polyurethanes, acetel materials, Teflon® materials, and the like; thermoset materials such as silicones; and materials that are otherwise curable, e.g., that can be cured by ultraviolet radiation or chemical reactions, including curable materials such as curable urethanes, epoxies, acrylates, cyanoacrylates, and the like. Any of these materials may be homopolymers, copolymers, or a blend or other combination of homopolymers, copolymers, or both. Other suitable synthetic materials include metals (e.g. silver filigree, tantalum gauze mesh, and stainless steel mesh).

Examples of specific synthetic film and mesh materials are known and may be suitable for use as a portion or piece of the stent. These include biocompatible materials that may be bioabsorbable or non-bioabsorbable, e.g., in the form of mesh materials. Suitable materials include cotton, linen, silk, polyamides (polyhexamethylene adipamide (nylon), polyhexamethylene sebacamide (nylon), polycapramide (nylon), polydodecanamide (nylon), and polyhexamethylene isophthalamide (nylon), and copolymers and blends thereof), polyesters (e.g., polyethylene terephthalate, polybutyl terephthalate, copolymers and blends thereof), fluoropolymers (e.g., polytetrafluoroethylene and polyvinylidene fluoride), polyolefins (e.g., polypropylene, including isotactic and syndiotactic polypropylene and blends thereof, as well as blends composed predominantly of isotactic or syndiotactic polypropylene blended with heterotactic polypropylene, and polyethylene), silicone, polygalactin, Silastic, polycaprolactone, polyglycolic acid, poly-L-lactic acid, poly-D-L-lactic acid and polyphosphate esters.

Commercial examples of polymeric materials for use in an implant include MARLEX (polypropylene) available from Bard of Covington, R.I.; PROLENE (polypropylene) and PROLENE Soft Polypropylene Mesh or Gynemesh (nonabsorbable synthetic surgical mesh), both available from Ethicon, of New Jersey; MERSILENE (polyethylene terephthalate) hernia mesh also available from Ethicon; GORE-TEX (expanded polytetrafluoroethylene) available from W. L. Gore and Associates, Phoenix, Ariz.; INTEPRO™ polypropylene materials, and the polypropylene material used in the commercially available MONARC™ or SPARC® sling systems, available from American Medical Systems, Inc. of Minnetonka, Minn. Commercial examples of absorbable materials include DEXON (polyglycolic acid) available from Davis and Geck of Danbury, Conn., and VICRYL available from Ethicon.

As described herein, the basic components of the stent include an internal frame and an external casing. The stent can comprise one or more layers of the external casing. For example, the stent can comprise 1 layer, 2 layers, 3 layers, 4 layers, 5 layers, 6 layers, 7 layers, 8 layers, 9 layers, 10 layers, 11 layers, 12 layers, 13 layers, 14 layers, 15 layers, 16 layers, 17 layers, 18 layers, 19 layers, 20 layers, 21 layers, 22 layers, 23 layers, 24 layers, 25 layers, or more than 25 layers. In an embodiments, the stent comprises between 4 layers and 15 layers of external casing. In embodiments the stent comprises no more than 15 layers of the external casing. In embodiments, the stent comprises 4 or more layers of the external casing. In embodiments, the stent comprises 8 layers of the external casing.

By varying the number of layers of the external casing, one can optimize the period of time during which the external casing degrades or dissolves. For example, fewer layers of external casing degrades faster than many layers of external casing. The skilled artisan will recognize that the rate at which the external casing degrades is dependent on the material of the external casing. For example, the stent can comprise 10 layers of external casing, which, in embodiments, degrades about 60 percent in 1 month, and is completely degraded in about 3 months.

By varying the thickness of the external casing, one can optimize the period of time during which the external casing degrades or dissolves. For example, the thicker the external casing, the longer the external casing takes to degrade. In embodiments, the external casing of the invention can comprise a thickness of about 1 mm to about 4 mm. For example, the thickness of the external casing can be less than 1 mm, about 1 mm, about 2 mm, about 3 mm, about 4 mm, or greater than 4 mm.

By varying the thickness of the internal frame, one can optimize the period of time during which the internal frame degrades or dissolves. For example, the thicker the internal frame, the longer the internal frame takes to degrade. The internal from of the invention can comprise a thickness of about 1 mm to about 2 mm.

The internal frame can be made of a material that is suitable for 3D printing, such as those described herein.

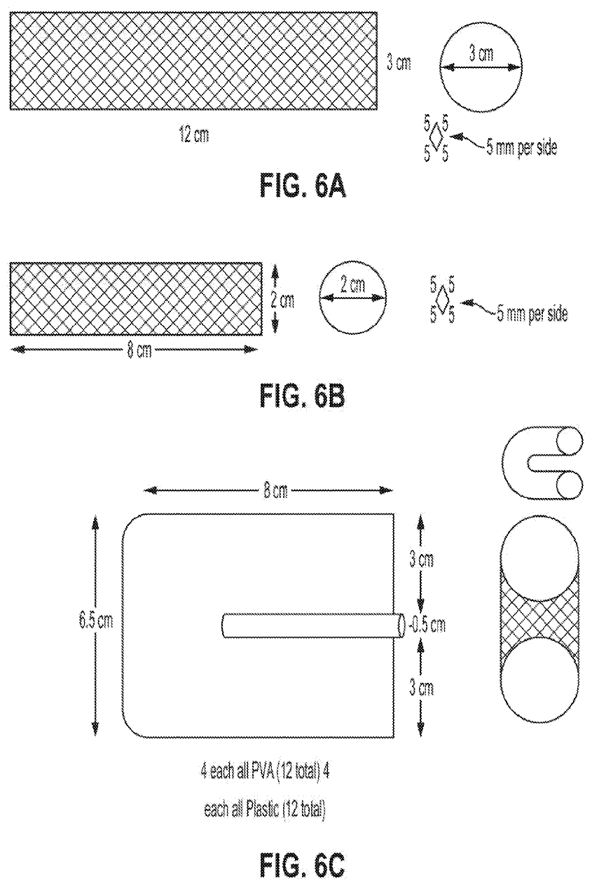

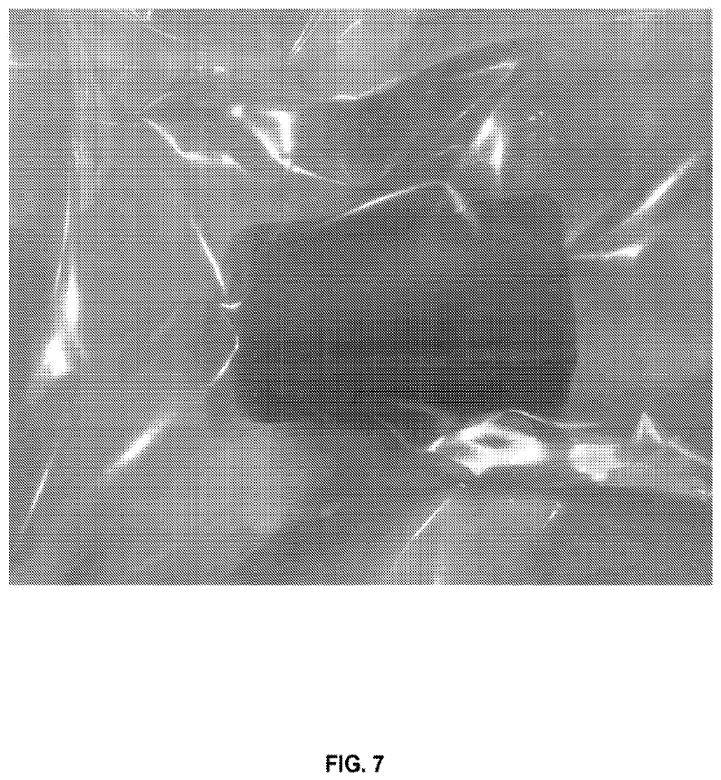

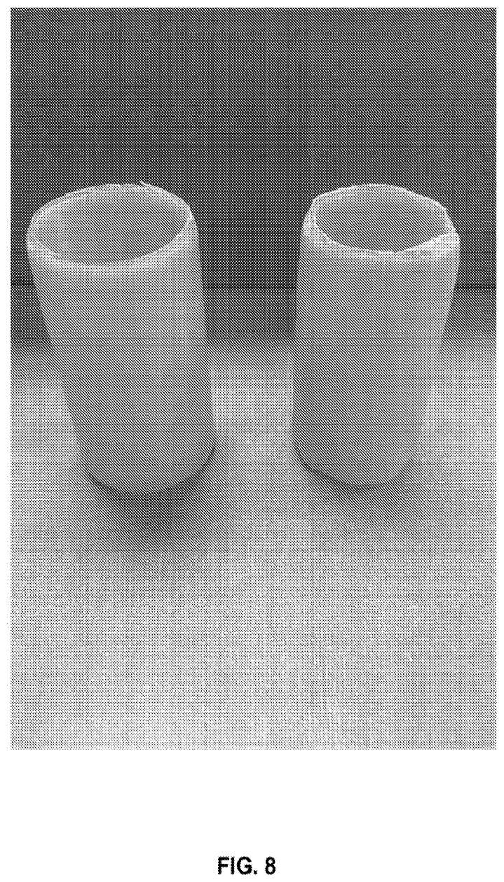

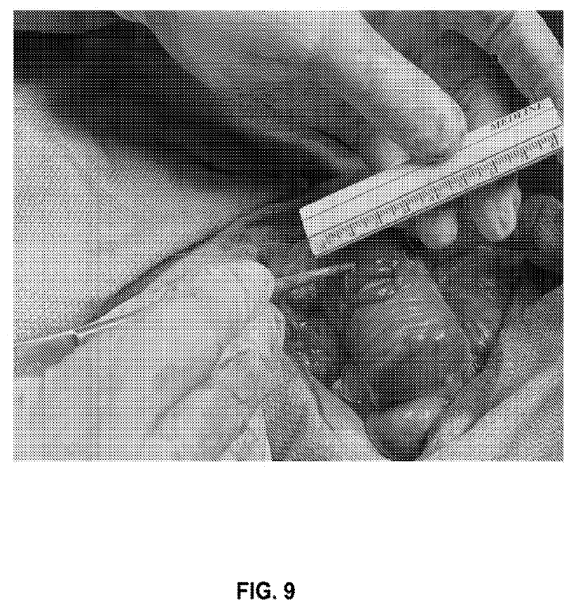

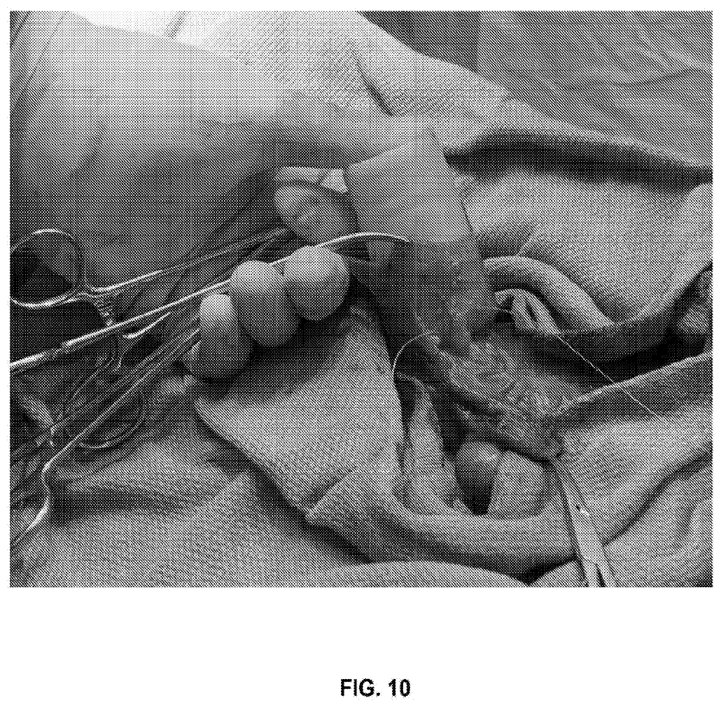

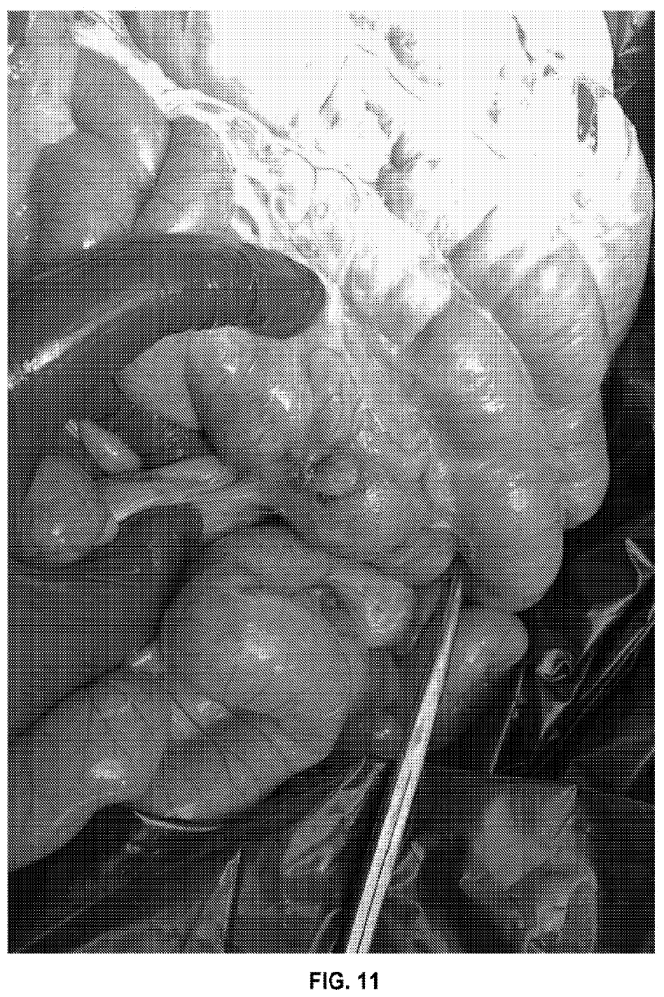

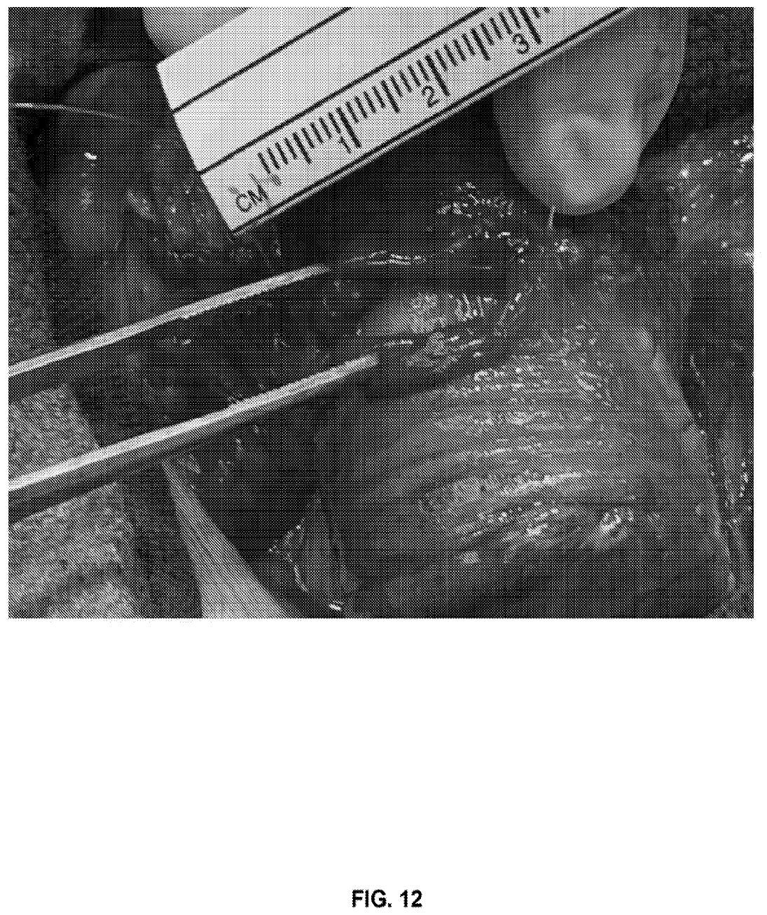

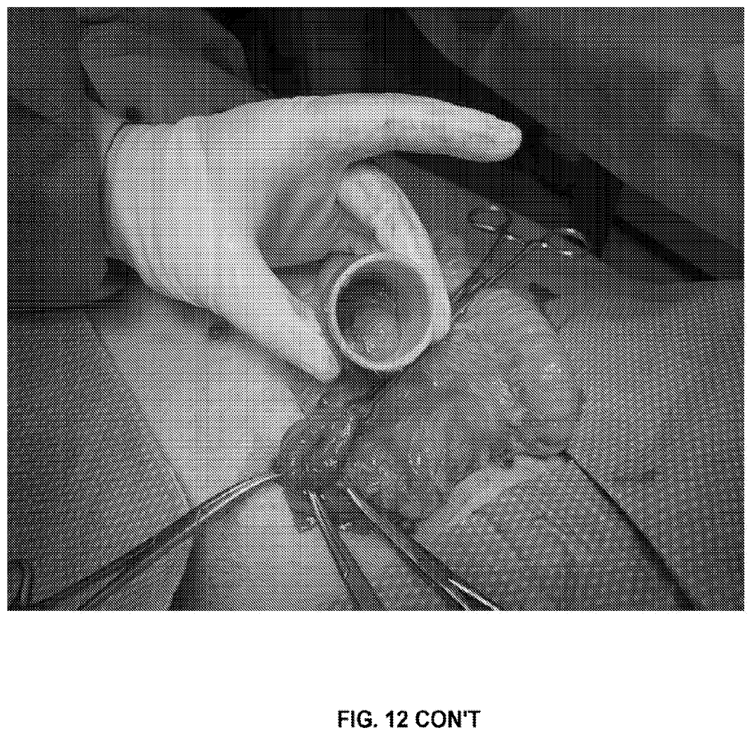

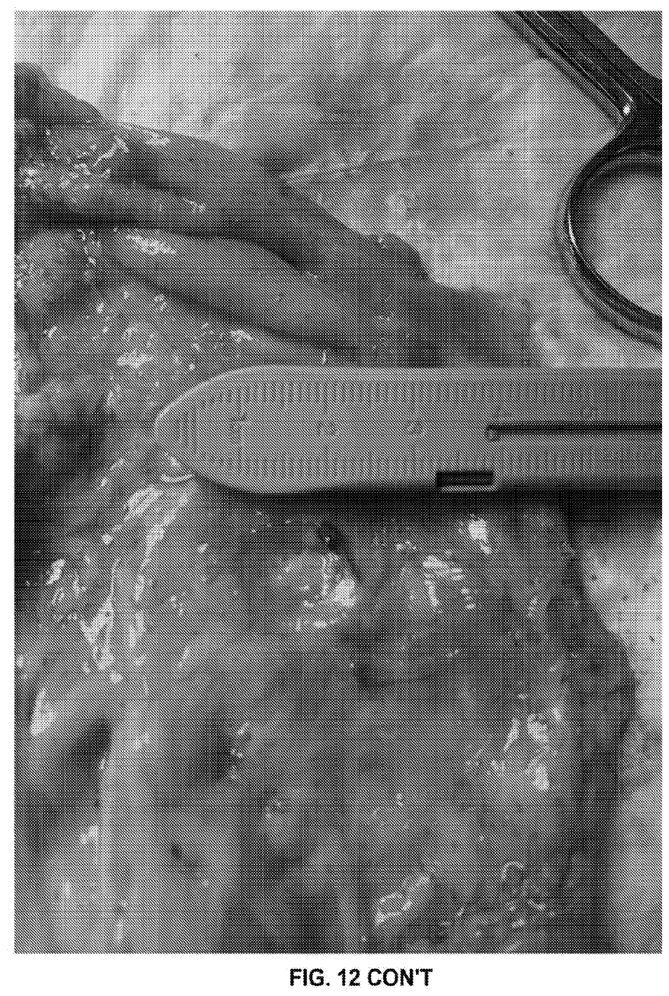

As described herein, the external casing can substantially cover the internal frame, and a cavity or lumen extends through the stent along a longitudinal axis. The diameter of the stent can depend on the site and size of the anastomosis. Exemplary schematics of various exemplary embodiments of the stent are shown in FIGS. 6A-6C. For example, the diameter of the stent can be between about 10 mm to about 80 mm. In one embodiment, the diameter is from about 22 mm to about 60 mm. In an embodiment, the diameter of the stent is about 32 mm. The diameter can be up to about 200 mm. In embodiments, the diameter can be less than about 10 mm.

Likewise, the length of the stent can depend on the site and size of the anastomosis. The length of the stent can be about 3 cm to about 50 cm. The length of the stent can range from about 10 cm to about 30 cm. In embodiments, the stent can be longer than 30 cm. In one embodiment, the stent can be, 3 feet or more. In some instances, the internal frame can span the entire length of the stent. Alternatively, the internal frame can simply comprise the two or more ends of the stent with the external casing comprising the ends of the stent and also the middle of the stent.

For example, an enteric stent can be about 6 to 10 cm in length, and about 20-30 mm in diameter.

As shown in FIG. 3F, in certain embodiments, the stent 700 can comprise one or more flanged ends 764. For example, the diameter of the stent body 762 can be 25 mm, and the diameter of the flanged ends 764 can be 30 mm.

Referring to FIG. 4, the stent can be configured to be affixed to a site of anastomosis. The term “anastomosis” is described herein.

The stent can be flexible, semi-flexible, or rigid. A rigid or semi-flexible stent, for example, can function as structural scaffold to keep either the subject's tissue, the external casing, or both, rigid or semi-rigid (but not floppy). Thus, the stent will allow a medical professional to more easily sew the rigid tissue, such as to attach the external casing to the site of anastomosis or to suture the anastomosis itself. For example, a rigid device comprising the stent can be placed through the mouth or anus and then the rigid device removed after stent is sewn through the bowel.

The stent of the invention can be dehydrated prior to implantation, such as to produce a stent capable of being stored for a period of time. For example, the external casing can be placed over an internal frame, thus forming a stent, and the stent can then be dehydrated for storage. Prior to implanting the stent into the subject, the dehydrated stent can be rehydrated with a solution, such as water or saline solution.

Referring to FIGS. 6A-F, the stent can be in a straight line or substantially straight (see 100, 300, 310, 600, and 700 of FIGS. 6A-B, E-F), or the stent can comprise a 10°, 20°, 30°, 40°, 50°, 60°, 70°, 80°, 90°,120°,150°,180° angle. For example, the stents of FIGS. 6C & 6D can comprise a 180° angle and be “C” shaped 400, 500, such as to turn anastomotic corners. In another embodiment, the stent can be “Y” shaped, such as cross end to side anastomosis. In embodiments, the stent is configured for use when the bowel is anastomosed side to side. In embodiments, the stent is configured for use when the bowel is anastomosed end to side.

The stent of the present invention can be configured to be implanted within any structure in the body that comprises a lumen, for example a structure in the gastrointestinal tract (i.e., enteric stent), lungs, trachea, esophagus, bile duct, vascular, lacrimal, ear, salivary gland, artery, veins, lymph ducts, dialysis, microsurgical, pancreatic duct, ureter, urethra, vas deferens, neurosurgical, nerve covering, tendon covering. Embodiments of the invention can also be used to treat bowel leaks, fistula, stricture, areas of potential weakness form endoscopic mass removal, rectal and anal fistula and sinuses. Embodiments of the invention can be used for covering J-pouch after total colectomy, and anastomosis after diverticulitis resection, and rectal trauma, to prevent colostomy. In addition to preventing anastomotic leaks, stents of the present invention can be used to reconstruct and/or repair any tissue with a lumen, such as bowels, esophagus, intestine, or trachea. Stents of the present invention can also provide the framework for growing new tissue (such as bowel lengthening) or tissue repair (such as bowel repair).

In embodiments, the stent can be configured to be a bioscaffold that is populated with viable cells or is capable of being populated by viable cells. Viable cells will promote healing of the site of anastomosis. The term “viable cell” can refer to a cell that is alive and capable of growth, proliferation, migration, and/or differentiation. For example, a tissue scaffold can comprise matrices, such as collagen matrix. Generally, the intestines comprise epithelial tissue comprising epithelial cells. For example, an enteric stent can be populated with epithelial cells prior to implantation, or the stent can be configured to be populated with epithelial call after implantation. For example, in some embodiments, cells from the native tissue (e.g., the host subject) can migrate into the stent and readily repopulate the stent (and thus promote healing). In embodiments, the stent can be seeded with viable cells so as to repopulate the stent with the viable cells prior to implantation.

Methods of Implanting the Anastomosing Stent

The present invention further comprises methods of implanting within a subject the stent of the present invention. For example, the method comprises obtaining a stent of the present invention; and implanting the stent to a site in the subject, thereby implanting in the subject the stent.

The site to which the stent is implanted can comprise a site of anastomosis. An anastomosis is a connection or opening between tubular structures. The anastomosis can be created by surgery, trauma, or disease. For example, a surgical anastomosis refers to a surgical technique used to make a new connection between two body structures that carry fluid, such as blood vessels or bowel. A surgical anastomosis can be created using suture sewn by hand, mechanical staplers and biological glues, depending on the circumstances. While an anastomosis may be end-to-end, equally it could be performed side-to-side or end-to-side depending on the circumstances of the required reconstruction or bypass.

Surgical anastomosis can be performed on structures in the GI tract, such as the esophagus, stomach, small bowel, large bowel, bile ducts, or pancreas. Virtually all elective resections, such as of gastrointestinal organs, are followed by anastomoses to restore continuity. For example, pancreaticoduodenectomy is considered a massive operation, in part, because it requires three separate anastomoses (stomach, biliary tract and pancreas to small bowel). Bypass operations on the GI tract, once rarely performed, are the cornerstone of bariatric surgery.

If there is a defect in the anastomosis, contents can leak out of the lumen of the structure and contaminate the surrounding cavity. Anastomotic leakage can occur in the intestine (i.e., colon), esophagus, stomach, rectum, bile duct, ureter, or urethra. For example, intestinal resection requires anastomosis, which if defective can cause an intestinal anastomotic leak. In an intestinal anastomotic leak, bowel content can leak out of the bowel and contaminate the normally sterile peritoneal cavity, causing peritonitis. Peritonitis (infection of the peritoneal cavity) can be lethal, and, therefore, measures must be taken during surgery to ensure that defects in the anastomosis are not present. The stents of the present invention can reduce the likelihood of or prevent anastomotic leakage and promote healing of the anastomosis.

Depending on the nature and site of the anastomosis, the stent can be implanted under the site of anastomosis or over the site of anastomosis. For example, when there is a long distance of bowel, a covering may be placed within the bowl ends and over the bowel to create a channel for new bowel to grow together. For example, embodiments may comprise a solid stent and covering on inside and/or solid covering only on outside, and cut ends of bowel in middle.

The stent 100 can be anchored to the anastomosis site or surrounding tissue by fasteners (251, 253 at FIGS. 4 & 5) known to the skilled artisan, such as by pressure, an adhesive (such as fibrin glue), a clip, a tack, a suture, a staple, or a screw.

As described herein, the stent can be rigid or semi-rigid, such as to allow for the anchoring of the stent into the body tissue. For example, the stent can be provided as a dry, rigid stent, and surgically implanted into the subject. In an alternative embodiment, the stent can be provided as a dehydrated stent which is moistened prior to implantation, so as to allow the stent to become pliable and implanted using an endoscopic balloon. The pliable covering can be placed over a collapsed expandable stent in the factory and dehydrated to make small enough to fit through endoscopic channel or into bowel. The entire stent can be rehydrated before placement to make pliable to expand. Once in place, the stent can be sewn into the existing tissue of the subject, or held in place by other means described herein. Over a period of time, the internal frame of the stent will first degrade, be absorbed, or dissolve, leaving the external casing membrane. Alternatively, the internal frame and the external casing can dissolve at same time. In embodiments, the external casing of the stent continues to prevent anastomotic leakage and promotes healing. The balloon and internal frame can be straight and tubular, flanged or not flanged, flanged at only the proximal side, or only covered with internal stent at the ends. The stent can be “C” shape to be placed down each limb of a side to side anastomosis, or the balloons can be “T” shaped to cover an end to side anastomosis.

Kits

Aspects of the invention are further directed towards kits comprising a stent(s) of the present invention and informational material.

Components of a multi-piece or multi-material stent can be pre-attached or pre-assembled in the kit, e.g., attached during manufacture, so a surgeon is not required to spend significant time cutting, connecting, or otherwise assembling the pieces of a stent prior to a surgical installation procedure. Alternatively, the components of the stent can be provided in two or more individual pieces, allowing the surgeon the option to build a stent that is configured to a specific tissue in the subject (i.e., personalized stent).

The kit can further comprise one or more solutions for rehydrating a dehydrated stent, and also means for affixing the stent to a subject's tissue, such as those means described herein.

The informational material can be descriptive, instructional, marketing or other material that relates to the methods described herein and/or the use of the stent for therapeutic benefit. The informational material of the kits is not limited in its form. In one embodiment, the informational material can include information about production of the stent, date of expiration, batch or production site information, and so forth. In one embodiment, the informational material relates to methods of implanting the stent. The information can be provided in a variety of formats, include printed text, computer readable material, video recording, or audio recording, or an information that provides a link or address to substantive material.

In addition to the stent, the composition in the kit can include other ingredients, such as a solvent or buffer, a stabilizer, or a preservative. When the stents are provided in a hydrated form, the stent can be provided in a liquid solution. The liquid solution can be, for example, an aqueous solution. When the stents are provided as a dried form, reconstitution generally is by the addition of a suitable solvent. The solvent, e.g., sterile water or buffer, can optionally be provided in the kit.

The kit can include one or more containers for the stents. In some embodiments, the kit contains separate containers, dividers or compartments for the stent and informational material. For example, the composition can be contained in an air tight, waterproof (e.g., impermeable to changes in moisture or evaporation), and/or light-tight pack, and the informational material can be contained in a plastic sleeve or packet. In other embodiments, the separate elements of the kit are contained within a single, undivided container.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Collagen Enteric Stent to Prevent Anastomotic Leak and Treat Fistula Both Tube Stent or Flat Patch

Surgical anastomosis, such as of the esophagus, intestine, stomach, colon and rectum, have a high incidence of leak after resection with rates between 1-30%. Leaks lead to death, morbidity, increased cost and ostomy.

Intestinal, rectal, and anal fistula between the skin and other organs have been treated with permanent stents, but these stents have to be removed, can migrate, and are not flexible enough to be placed around tight corners. A absorbable patch or tube can use the same technology to treat these diseases. Placing a slowly absorbable stent would prevent the unwanted side effects by preventing leakage while the anastomosis heals without the issues of a permanent stent. The collagen tubes used in validation studies are 32 mm diameter and can be folded on themselves to increase the layers.

There has never been a treatment which has prevented anastomotic leaks, and surgeons, patients, and insurance companies would be very eager to use a product that solved this issue. There is not a biologic enteric stent on the market. Processed collagen is an inexpensive material that has sufficient strength to prevent leakage but is flexible. The stent would be deployable with a balloon and/or sutured with slowly absorbable PDS sutures.

Additional validation studies in animal will be conducted. Studies may utilize a porcine model with esophageal, small bowel and colon anastomosis with only 4 sutures and large gaps in the tissue which will leak. There will be a stent sewn within the anastomosis to prevent leakage. There will also be bowel anastomosis with a complete gap in to bowel not connecting at all, with only the stent covering within on over, to evaluate if the bowel can grow in length with the stent internal and external covering as a channel.

### Example 2

### Biologic Enteric Stent for the Prevention of Anastomotic Leaks

Summary: An exemplary embodiment comprises a hybrid enteric stent created with an internal frame, such as a Polyvinyl Alcohol (PVA) internal frame, and an external casing, such as a porcine submucosa covering. A patch (i.e., not a complete covering) of acellular porcine submucosa has been used to repair bowels and hernia in animals and humans. Previous studies have shown that when acellular porcine graft is placed next to living human tissue, the body uses it as a scaffold to grow, creating a stronger repair mechanism than using sutures alone. Without wishing to be bound by theory, this stent will keep intestinal contents from leaking through the bowel as it heals, and that the stent will degrade via the enteric contents.

Background: Bowel resection is a common surgical procedure. More than one million bowel resections are performed annually in the United States alone: 600,000 colon resections preformed per year in 2015 (SAGES Data), 400,000 new esophageal cancers diagnosed per year, 179,000 gastric bypass procedures performed in 2013, and small bowel resection is common in trauma, inflammatory bowel disease, adhesions, fistula, and bowel obstruction conditions. Bowel resection is also used to treat many congenital conditions within the pediatric population. The leak rate from bowel anastomosis is between 3-26%, with $28.6 million in additional cost per 1,000 post-op colon resection patients in the first 30 days with leaks (4). It is a conservative estimate that hundreds of millions of dollars are spent annually to address the complications of bowel leaks.

Enteric anastomosis are common procedures for thoracic, general, laparoscopic, and colorectal surgeons. Leaks from esophageal, small bowel and colon are a major source of morbidity and mortality with a mortality rate of 6-39% (1). In addition to death, leaks lead to sepsis, abscesses, fistulas, and the anastomosis being taken down and an ostomy being performed. Some patients are at such a high risk for enteric leak that creating an ostomy is a safer procedure. One common example is perforated diverticulitis, and these patients live with ostomies for months to years. Collectively, these interventions add a major financial cost to patients, hospitals, and the health care system.

For the last 100 years, placing mesh has been the standard of care for repairing a hernia. Synthetic mesh was the first generation of this technique, and the second generation advanced to biologic mesh made of porcine or human dermis. Acellular biologic materials have lower complication rates because they incorporate into native tissue. The technique of creating bowel anastomosis has not advanced accordingly, and surgeons are currently only using sutures or staples without any reinforcement and accepting leaks, fistula, and ostomies. Therefore, the stent herein addresses an unmet medical need and represents a very large market opportunity.

Aspects of the invention: Aspects of the invention comprise a hybrid enteric stent created with an internal frame, such as a Polyvinyl Alcohol (PVA) internal frame, and/or an external casing, such as a starched Polyglactin (Vicryl) with a porcine submucosa covering. PVA is poorly absorbed by the GI tract and is nontoxic; it used as a coating for pills and as a wash for contact lenses. PVA also has the benefit of being a material currently used in 3D printers. Vicryl sutures have been used for decades and are safe; they can be placed in the bowel and have the capacity for reabsorption. Porcine submucosa is the strength layer of bowels; acellular porcine submucosa is available on the U.S. market for use in eye repair surgery, hernia repair, and is successful in treating rectal fistula and as a patch for bowel repair.

Embodiments of the invention can be created using a Vicryl tube and then covering the stent with porcine submucosa. Referring to FIG. 4, for example, this stent will then be placed inside to cover the bowel anastomosis, repair, or potential weakness, which will be reconnected with, anchored to, and, ideally, incorporated into the inside of the anastomosis with sutures. When acellular porcine graft is placed next to living human tissue, the body uses it as a scaffold to grow, creating a stronger repair mechanism than using sutures alone. Without wishing to be bound by theory, the stent will keep the intestinal contents (stool) from leaking through the bowel as it heals, and then the stent will degrade via the enteric contents. Acellular porcine submucosa has been used to repair bowels and hernia in animals and humans, and has been shown to be strong enough to resist enzymatic degradation. Without wishing to be bound by theory, our stent will be able to limit or minimize leakage, and thus reduce costs allocated annually to treating leaks and relevant complications, and also potentially save thousands of lives per year.

In some patients, there is such a high risk of leak at the time of surgery, that the surgeon brings up an ostomy instead of connecting the bowel. Patients must collect stool into a bag attached to their abdomen instead of having normal bowel movements. These patients will have to live with ostomies for months to years, until they undergo another surgery to reconnect their bowels or, in some cases, the rest of their lives. Having to live with an ostomy is a huge burden and fear for patients. If it was possible to place a stent at the time of surgery, instead of creating an ostomy, it would improve the lives of many patients.

Embodiments of the stent herein will be applicable to companies that manufacture from synthetic stents and biologic grafts. This device will also be applicable to surgeons, as it will prevent their patients from complications related to a leak or ostomy.

Porcine submucosa is currently sold for a patch and plug for fistula. Other stent devices have been utilized in previous studies and produced mixed results. All previous stents have had the same permeant material technology (2). Purely biologic materials, such as amniotic membrane, have been utilized in an effort to recruit growth factors, but these materials lack structural elements, are expensive, and lack compelling evidence that they are effective.

Permanent enteric stents, which have to be placed with an endoscope, have been on the market for years and are effective in treating bowel stenosis and leaks; however, they are only placed after a complication has developed, and there are locations in the bowel (such as corners) where they cannot be placed.

Endoscopic stents can themselves cause secondary complications, and need to be placed and retrieved with endoscopy. There is an absorbable synthetic stent (ELLA-CS) that effectively treats leaks, but it has negative issues with migration and the covered version has a silicon covering that prevents ingrowth of tissue; thus, it is not designed to be placed at the time of surgery.

There are absorbable stents currently on the market, but none have been placed at the time of surgery to prevent leaks, and none have used a biologic material as a scaffold for tissue ingrowth (7). Retrievable permanent stents have been on the market for years, and they have demonstrated profitability.

There are also biologic meshes, such as an acellular porcine submucosa Surgisis for bowel patch.

Acellular materials have been used in humans for over 20 years and are easily obtainable and inexpensive.

Another benefit of the porcine stent is that other experiments have shown that native tissue can grow along a biologic scaffold (3). One arm of the study will be to create a gap in native tissue to see if the bowel will grow in length and function. This could lead to this graft being used to treat children and adults with short gut syndrome. Currently, there are limited options for the treatment of short gut syndrome, and patients live on intravenous nutrition or undergo intestinal transplant.

Validation Studies:

Referring to FIGS. 1 and 4, collagen stents have been prepared, which have been sewn into cadaveric porcine intestine.

Embodiments can comprise 3D printed Polyvinyl Alcohol scaffold, for example, which can be printed into straight tubes for the esophagus, small bowel, and colon. Phalange ended stents for anastomosis with size mismatches, and 180 degree curves for side-to-side anastomosis can also be printed.

Vicryl mesh is also easily obtainable. No stent on the market has these components or configurations.

Validation studies will include animal studies, which will include surgically placing the stents into small bowel and colon anastomoses and then sacrifice the animals at weekly intervals to evaluate if the stents prevented leaks and quantify the degradation and incorporation of the material into the native tissue.

### REFERENCES CITED IN THIS EXAMPLE

1. Zuri A. M., Stamos M. Reoperation for Anastomotic Failure. Clin. Colon Rectal Surg. 2006 November; 19(4): 213-216.
2. Morks A N, Havenga K, Ploeg R J. Can intraluminal devices prevent or reduce colorectal anastomotic leakage: a review. World J Gastroenterol. 2011 Oct. 28; 17(40):4461-9.
3. Pramod P. REGENERATION OF FUNCTIONAL BLADDER SUBSTITUTES USING LARGE SEGMENT ACELLULAR MATRIX ALLOGRAFTS IN A PORCINE MODEL. The Journal of Urology, Volume 164, Issue 3, Part 2, September 2000, Pages 941
4. Hammond, Sangtaeck. The Burden of Gastrointestinal Anastomotic Leaks: an Evaluation of Clinical and Economic Outcomes. J Gastrointest Surg. 2014; 18(6): 1176-1185.
5. Huang-Chien Lianga, Yen Chang, Effects of crosslinking degree of an acellular biological tissue on its tissue regeneration pattern. Biomaterials 25 (2004) 3541-3552
6. Downey D M I, Harre J G, Dolan J P.Increased burst pressure in gastrointestinal staple-lines using reinforcement with a bioprosthetic material.
7. Anthony Clough MBBS, Porcine Small Intestine Submucosa Matrix (Surgisis) for Esophageal Perforation.

### Example 3

### Preliminary 2-Week Data From Pig Study

We sewed 10 PVA/porcine small bowel submucosa 8 layer of SIS stents into the colon of 10 pigs. We left 2 cm defects in the anastomoses over the stents. Previous studies have shown that leaving a 2 cm hole in the colon of a pig will result in a 100 percent leak rate. See, for example, Rosenberger, Laura H., et al. “Delayed endoluminal vacuum therapy for rectal anastomotic leaks after rectal resection in a swine model: a new treatment option.” Clinical and translational science 7.2 (2014): 121-126, and Nordentoft, Tyge, and Michael Sorensen. “Leakage of colon anastomoses: development of an experimental model in pigs.” European Surgical Research 39.1 (2007): 14-16.

At 2 weeks post op we removed the colon and dissected the area. There were no signs of leak or abscess or fistula. All the holes had healed well and were strong. The stent was in the process of degrading. The stents cause no obstruction, bleeding or any complications at all. All pigs ate well.

This shows that a biodegradable stent of any kind and especially PVA/porcine submucosa stent can prevent leaks in colon up to 2 cm.

The stent utilized in the 2-week study was of the following dimensions: 1) Length-6 cm 2) Diameter-3.5 cm 3) Thickness-1.5 mm.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. An anastomosing stent comprising an internal frame and an external casing, wherein the external casing substantially covers the internal frame, and the stent further comprises a cavity extending therethrough along a longitudinal axis.

2. The stent of claim 1, wherein the stent substantially or completely dissolves over a period of time when implanted in a subject.

3. The internal stent of claim 2, wherein the period of time comprises about 3 months.

4. The stent of claim 2, wherein the internal frame dissolves in about 24 hours.

5. The stent of claim 2, wherein the external casing dissolves in about 1 month.

6. The stent of claim 1, wherein the stent comprises one or more layers of the external casing.

7. The stent of claim 6, wherein the stent comprises no more than 20 layers of the external casing.

8. The stent of claim 7, wherein the stent comprises between 4 layers and 15 layers of the external casing.

9. The stent of claim 7, wherein the stent comprises no more than 15 layers of the external casing.

10. The stent of claim 7, wherein the stent comprises 4 or more layers of the external casing.

11. The stent of claim 7, wherein the stent comprises 8 layers of the external casing.

12. The stent of claim 1, wherein the thickness of the internal stent comprises about 1 mm to about 2 mm.

13. The stent of claim 1, wherein the thickness of the external casing comprises about 1 mm to about 4 mm.

14. The stent of claim 1, wherein the internal frame is constructed of a biocompatible material.

15. The stent of claim 14, wherein the biocompatible material is a non-toxic material.

16. The stent of claim 14, wherein the biocompatible material comprises a biodegradable material which substantially or completely dissolves over a period of time when implanted in a subject.

17. The stent of claim 16, wherein the period of time comprises no more than about 2 hours.

18. The stent of claim 16, wherein the period of time comprises no more than about 24 hours.

19. The stent of claim 16, wherein the period of time comprises no more than about 3 months.

20. The stent of claim 14, wherein the material comprises polyvinyl alcohol (PVA), starched polyglactin (vicryl), collagen, magnesium, plant based fiber, or any combination thereof.

21. The stent of claim 14, wherein the material comprises an inert material.

22. The stent of claim 21, wherein the inert material comprises a fiber.

23. The stent of claim 22, wherein the fiber comprises a plant based fiber.

24. The stent of claim 1, wherein the internal frame is 3D printed.

25. The stent of claim 1, wherein the external casing is constructed of a biocompatible material.

26. The stent of claim 25, wherein the biocompatible material is a non-toxic material.

27. The stent of claim 25, wherein the biocompatible material comprises a biodegradable material which substantially or completely dissolves over a period of time when implanted in a subject.

28. The stent of claim 27, wherein the period of time comprises about 3 months.

29. The stent of claim 25, wherein the material comprises a biological membrane.

30. The stent of claim 29, wherein the biological membrane comprises an acellular or substantially acellular biological membrane.

31. The stent of claim 29, wherein the biological membrane comprises a decellularlized or substantially decellularized biological membrane.

32. The stent of claim 29, wherein the biological membrane comprises mammalian connective tissue and/or basement membrane.

33. The stent of claim 32, wherein the connective tissue comprises mammalian submucosa or amniotic membrane.

34. The stent of claim 33 wherein the mammalian submucosa comprises biological membrane comprises porcine submucosa, human submucosa, or bovine submucosa,

35. The stent of claim 29 wherein the biological membrane comprises dermis, pericardium, blood vessel, or plant-based material.

36. The stent of claim 25, wherein the material comprises a synthetic material.

37. The stent of claim 25, wherein the material comprises an absorbable material.

38. The stent of claim 25, wherein the material comprises a non-porous material.

39. The stent of claim 1, wherein the stent comprises an enteric stent.

40. The stent of claim 1, wherein the stent is configured to be affixed to a site of anastomosis.

41. The stent of claim 1, wherein the diameter of the stent is about 22 mm to about 60 mm.

42. The stent of claim 1, wherein the diameter of the stent is about 32 mm.

43. The stent of claim 1, wherein the length of the stent is about 10 cm to about 30 cm.

44. The stent of claim 1, wherein the stent is flexible, semi-flexible or rigid.

45. The stent of claim 1, wherein the stent is “C” shaped.

46. The stent of claim 1, wherein the stent is “Y” shaped.

47. The stent of claim 1, wherein the stent is configured to be a bioscaffold.

48. The stent of claim 47, wherein the bioscaffold is populated with viable cells.

49. The stent of claim 48, wherein the viable cells comprise epithelial cells.

50. A method of implanting within a subject the stent of claim 1, comprising:
obtaining the stent of claim 1; and
implanting the stent to a site in the subject, thereby implanting in the subject the stent.

51. The method of claim 50, wherein the site comprises anastomosis.

52. The method of claim 51, wherein the site comprises surgical anastomosis.

53. A method for preventing anastomotic leakage comprising implanting within a subject known to have or at risk of having an anastomosis the stent of claim 1, thereby preventing leakage.

54. The method of claim 53, wherein the stent is implanted under the site of anastomosis, implanted to cover leak, fistula, stricture.

55. The method of claim 53, wherein the stent is implanted over the site of anastomosis.

56. The method of claim 54 or 55, wherein the stent is affixed to the site of anastomosis by pressure, sutures, or clips.

57. The method of claim 54, wherein the anastomotic leakage is in the intestine (i.e., colon), esophagus, stomach, rectum, bile duct, ureter, or urethra.

58. The method of claim 54, wherein the cause of the anastomotic leakage is surgery.

59. The method of claim 58, wherein the surgery comprises tissue resection.

60. The method of claim 50 or claim 53, wherein the stent is implanted using an endoscopic balloon.

61. A method of making a stent comprising:
obtaining an internal frame and an external casing, wherein the external casing comprises a biological membrane; placing the external casing over the internal frame, wherein the external casing substantially covers the internal frame, wherein a cavity extends through the internal frame along a longitudinal axis;
dehydrating the biological membrane to produce a stent capable of being stored for a period of time.

62. The method of claim 61, wherein the dehydrated stent is rehydrated in water or saline solution prior to implanting in the subject.

63. A kit comprising the stent of claim 1.

64. The kit of claim 63, wherein the kit comprises an internal frame and an external casing.

65. The kit of claim 64, wherein the external casing comprises a biological membrane.

66. The kit of claim 63, further comprising a solution for rehydrating the biological membrane and/or stent.
